(19) United States
(12) Patent Application Publication
(10) Pub. No.: US 2006/0224032 A1
(43) Pub. Date: Oct. 5, 2006

(54) Protecting catalytic sites of activated porous molecular sieves

(76) Inventor: Marcel J. Janssen, Kessel-Lo (BE)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 11/092,825

(22) Filed: Mar. 29, 2005

(51) Int. Cl. C07C 1/00; B01J 20/34; B01J 38/52

(52) U.S. Cl. 585/639; 502/33

(57) ABSTRACT

Metalloaluminophosphate molecular sieves and metalloaluminophosphate molecular sieve catalyst particles are protecting from degradation by water by maintaining said molecular sieves or catalysts in contact with a vapor and/or liquid mixture of aldehyde and at least 0.1 wt. % water and the activated porous molecular sieve has at least 80% of the pore volume filled with the agent. The metalloaluminophosphate molecular sieves and metalloaluminophosphate molecular sieve catalyst particles which have been protected in such fashion catalyze the conversion of feedstocks to hydrocarbons.

### FIELD OF THE INVENTION

This invention relates to a method of maintaining molecular sieves during storage and handling, to stabilized molecular sieves and stabilized molecular sieve containing catalysts and to the use of these stabilized molecular sieves or catalysts in adsorption and conversion processes.

### BACKGROUND OF THE INVENTION

Olefins are commonly produced from petroleum feedstock by catalytic or steam cracking processes. These cracking processes, especially steam cracking, produce light olefin(s) such as ethylene and/or propylene from a variety of hydrocarbon feedstock. It has been known for some time that oxygenates, especially alcohols, are convertible into light olefin(s). Methanol, the preferred alcohol for light olefin production, is typically synthesized from the catalytic reaction of hydrogen, carbon monoxide and/or carbon dioxide in a methanol reactor in the presence of a heterogeneous catalyst. The preferred methanol conversion process is generally referred to as a methanol-to-olefin(s) process, where methanol is converted to primarily ethylene and/or propylene in the presence of a molecular sieve.

Some of the most useful molecular sieves for converting methanol to olefin(s) are the metalloaluminophosphates such as the silicoaluminophosphates (SAPO's) and the aluminophosphates (AlPO's). SAPO synthesis is described in U.S. Pat. No. 4,440,871, which is herein fully incorporated by reference. SAPO is generally synthesized by the hydrothermal crystallization of a reaction mixture of silicon-, aluminum- and phosphorus-sources and at least one templating agent. Synthesis of a SAPO molecular sieve, its formulation into a SAPO catalyst, and its use in converting a hydrocarbon feedstock into olefin(s), particularly where the feedstock is methanol, is shown in U.S. Pat. Nos. 4,499,327; 4,677,242; 4,677,243; 4,873,390; 5,095,163; 5,714,662 and 6,166,282, all of which are herein fully incorporated by reference.

Metalloaluminophosphate molecular sieves contain a pore system, which is a network of uniform pores and empty cavities. These pores and cavities catch molecules that have a size equal to or less than the size of the pores and cavities, and repel molecules of a larger size.

The pores and cavities of molecular sieves are formed during the molecular sieve manufacturing process. It may be a result of adding template materials during the molecular sieve manufacturing process. During the formation of the molecular sieves themselves, a lattice type chemical structure forms around the template material, with the template material acting as a means of forming the pore structure within the molecular sieve. The resulting molecular sieve may be combined with other components for the benefit of adjusting various properties of the molecular sieve or to form larger particles.

To make the molecular sieve suitable for use, the template must be at least partially, preferably completely, removed so that the pores and cavities are open to catch molecules, either for the purpose of adsorbing the molecules from the environment or to react the molecules to form a desired product. The reaction occurs when the molecules come into contact with catalytic sites located within the pore system, particularly within one or more of the empty cavities or cages as sometimes called.

The template is conventionally removed from the molecular sieve by calcining or burning out the template. An elution process can also be used to remove the template, although calcination is preferred. Once the template is removed, the molecular sieve is considered to be activated or ready for use. The activated molecular sieve has its pore system, including the empty cavities or cages open to the immediate environment, and ready for use.

Activated metalloaluminophosphate molecular sieves that have catalytic sites within their microporous structure, e.g., silicoaluminophosphate (SAPO) molecular sieves, have been found to be sensitive to moisture. In general, significant exposure of the activated molecular sieves to moisture can deactivate the catalytic activity of the molecular sieves. Unfortunately, methods of protecting activated metalloaluminophosphate molecular sieves against the harmful effects of moisture are limited.

U.S. Pat. No. 6,316,683 (Janssen et al.) discloses a method of protecting catalytic activity of a SAPO molecular sieve by shielding the internal active sites of the molecular sieve from contact with moisture using an anhydrous blanket as a shield for an activated molecular sieve after removal of the template. The anhydrous blanket contains less than about 200 ppm water, preferably less than about 100 ppm water, more preferably less than about 50 ppm water and is preferably selected from the group consisting of alkanes, cyclo-alkanes, C₆-C₃₀ aromatics, alcohols, particularly C₄⁺ branched alcohols.

U.S. patent application Ser. No. 10/704,741, filed Nov. 10, 2003 (Janssen et al.), discloses a method of aging an activated porous metalloaluminophosphate molecular sieve, which is maintained in contact with a vapor and/or liquid mixture of alcohol and water, the mixture of alcohol and water containing from 45 wt. % to 99.8 wt. % alcohol.

U.S. patent application Ser. No. 10/712,952, filed Nov. 12, 2003, (Teng et al.), discloses a method of pretreating a fresh or regenerated metalloaluminophosphate molecular sieve, which is low in carbon content with an aldehyde. The aldehyde forms a hydrocarbon co-catalyst within the pore structure of the molecular sieve, and the pretreated molecular sieve containing the co-catalyst is used to convert oxygenate to an olefin product containing a significant amount of ethylene and propylene, with relatively low amounts of undesired by-products.

As new large scale commercial production facilities that use metalloaluminophosphate molecular sieves in the production process continue to be implemented, protecting the activated porous metalloaluminophosphate molecular sieves from loss of catalytic activity as a result of contact with moisture continues to become an even greater challenge. The present invention provides a new method of protecting the catalytic sites of activated porous metalloaluminophosphate molecular sieves or catalysts containing metalloaluminophosphate molecular sieves during molecular sieve storage and/or handling or during catalyst storage and/or handling.

### SUMMARY OF THE INVENTION

In one embodiment, the present invention relates to a method of maintaining the catalytic activity of an activated porous molecular sieve by contacting it with an agent comprising aldehyde and at least 0.1 wt. % water and the activated porous molecular sieve has at least 80% of the pore volume filled with the agent.

In another embodiment, the present invention relates to a method for converting an oxygenate feedstock into a hydrocarbon product by contacting the oxygenated feedstock under oxygenate to olefins conversion conditions with an activated porous molecular sieve having a methanol uptake index of at least 0.15, wherein, prior to contacting the feedstock, the catalytic activity of the molecular sieve catalyst is maintained by contacting the catalyst with an agent comprising aldehyde and at least 0.1 wt. % water.

In another aspect of each of the above embodiments, the activated porous molecular sieves or activated porous molecular sieve catalyst particles are maintained with a mixture of aldehyde and water containing at least 55 wt. % aldehyde, preferably, 80 wt. % aldehyde, more preferably at least 85 wt. % aldehyde, more preferably at least 90 wt. % aldehyde, and more preferably at least 95 wt. % aldehyde. In yet another aspect of each of the above embodiments, the activated porous molecular sieves or activated porous molecular sieve catalyst particles are maintained with an agent comprising at least 80 wt. % protective material and 0.1 wt. % water, wherein the protective material comprise at least 20 wt. % aldehyde, 0 to 40 wt. % alcohol, and a component have C₁ to C₁₂ hydrocarbons, and any combination thereof. Examples of C₁ to C₁₂ hydrocarbons are paraffins, olefins, and aromatics.

In yet another aspect of each of the above embodiments, the mixture of aldehyde and water contains from at least 0.1 wt. % to less than 20 wt. % water.

In yet another aspect of each of the above embodiments, the activated porous molecular sieves or activated porous molecular sieve catalyst particles are maintained by contacting with the mixture of aldehyde and water for at least 1 hour, preferably, at least 6 hours, more preferable, at least 12 hours, even more preferably, at least 24 hours.

In other embodiments, each of the above embodiments is further defined by selecting the aldehyde in said aldehyde and water mixture from the group consisting of alkyl aldehydes, the alkyl group having from 1 to 16 carbon atoms, more preferably from 1 to 5 carbon atoms, and mixtures thereof. In additional embodiments, the aldehydes further have a kinetic diameter less than the pore diameter of the porous molecular sieve. A preferred aldehyde in any of the above embodiments is acetyl aldehyde.

In other embodiments of the present invention, the amount of pore volume of the activated porous molecular sieve catalyst of any of the above embodiments occupied by the mixture of aldehyde/water is at least 80%, more preferably at least 85%, even more preferably at least 90, most preferably at least 95%.

Preferably, the activated porous molecular sieve of the above embodiments is used directly after it is contacted with the mixture of aldehyde and water to form an integrated hydrocarbon co-catalyst within the porous framework. In yet other embodiments of the present invention, the activated porous molecular sieve is pretreated under conditions suitable to form an integrated hydrocarbon co-catalyst within the porous framework after contact with aldehyde and water instead of using directly in an oxygenate to hydrocarbon process.

In further embodiments, the activated porous molecular sieves or activated porous catalyst particles of the above embodiments maintain a methanol uptake index of at least 0.5 during and after contact with the mixture of aldehyde and water.

Preferably, the activated porous molecular sieve of any of the above embodiments is an activated porous metalloaluminophosphate molecular sieve. More preferably, the activated porous metalloaluminophosphate molecular sieve is selected from SAPO-11, SAPO-17, SAPO-18, SAPO-34, SAPO-35, SAPO-37, SAPO-44, SAPO-56, metal containing forms thereof and intergrown forms thereof.

### DETAILED DESCRIPTION OF THE INVENTION

### 1. Introduction

Metalloaluminophosphate, especially SAPO, catalysts are susceptible to changes (generally, deterioration) in catalytic activity and structural changes as a result of continued exposure to even low levels of moisture. We have found that activated SAPO molecular sieves lose catalytic activity when the catalytic sites are exposed to an open-air environment for as little as a few hours, and that loss of catalytic activity becomes irreversible after a certain point.

The possibility of irreversible loss of catalytic activity presents a problem in the commercial production-to-use chain where storage, handling and transport of the molecular sieve and catalyst can be over a relatively long period of time. For example, the as manufactured molecular sieve can be stored or transported anywhere from 12 hours to many months, even as long as one year, before its final use as an activated catalyst. Even partial loss of catalytic activity is of particular concern in large-scale catalytic processes. As defined herein, a large scale catalytic process is one having at least 50 kg of catalyst, particularly one having at least 500 kg of catalyst, especially one having at least 5000 kg of catalyst.

The activated porous molecular sieve can also be protected from loss of catalytic activity through contact with water or water vapor by coating the individual molecular sieve particle or formulated catalyst particles or spheres with a polymer or wax to seal the external surface and prevent moisture ingress. This is preferably done when the particles are hot, preferably just after calcination. A well known technique for polymer coating objects to prevent exposure to moisture or other materials is to heat the object, then expose it to solid polymer, e.g., polyethylene, which melts and evenly coats the solid object. Very fine polymer particles could be sprayed onto the catalyst particles as it exists the calciner to result in a very thin layer of polymer on the catalyst. This would then protect the catalyst during storage in shipping containers and ultimately during transfer to plant storage drums and finally provide protection in the plant storage bins from even very low levels of moisture in the gas used to “inert” these storage bins. The coating could just be on the external catalyst surface or in the catalyst macropores as well.

### 2. Water and Aldehyde Mixtures

According to the present invention, the catalytic activity of activated metalloaluminophosphate molecular sieves or catalysts comprising metalloaluminophosphate molecular sieves are maintained by contacting the molecular sieves or catalysts with an agent comprising aldehyde and at least 0.1 wt. % water.

As defined herein, the aldehyde and water mixture in the present invention is a mixture of aldehyde and at least 0.1 wt. % water. Suitable aldehyde and water mixtures include vapor and/or liquid mixtures of aldehyde and water containing from 55 wt. % to 99.9 wt. % aldehyde, preferably, from 80 wt. % to 99.9 wt. % aldehyde, even preferably from 85 wt. % to 99 wt. %, most preferably from 90 wt. % to 99 wt. % aldehyde. Preferably, the aldehyde and water mixture contains from 0.1 wt. % to 20 wt. % water, more preferably from 0.1 wt. % to 10 wt. % water, even more preferably from 0.1 wt. % to 5 wt. %, most preferably from 0.1 wt. % to 2 wt. % water.

The water and aldehyde mixture may also contain other components in an amounts less than about 45 wt. %, such as other oxygenated compounds (e.g., ketones, alcohol, carboxylic acids, carboxylic esters, peroxides, epoxides, ethers) or alkanes.

While any aldehyde may be used, preferred aldehyde includes alkyl aldehyde. More preferably, the aldehyde is one or several alkyl aldehydes in which the alkyl group, which may be linear or branched, has from 1 to 16 carbon atoms, even more preferably from 1 to 9 carbon atoms and most preferably from 1 to 5 carbon atoms. Even more preferably, the aldehyde is one having an average kinetic diameter smaller than the pore diameters of the molecular sieve so than the aldehyde may diffuse inside the channels of the molecular sieves. From practical and economic points of view, the aldehyde is preferably acetyl aldehyde. Mixture of different alkyl aldehydes can also be used in this invention. Commercially available vapor and/or liquid mixtures of aldehyde and water are suitable for the present invention. Examples of commercial vapor and/or liquid mixtures of aldehyde and water include crude aldehydes, or any other grades of aldehyde, which typically contain about 1 wt. % levels of water.

When mixing aldehyde and water, various grades of water can be used, including demineralized water, boiler feed water, or purified process water. The water should not contain more than 0.5 wt. %, preferable no more than 0.1 wt percent, more preferably not more than 0.01 wt. % contaminants.

The mixture of aldehyde and water is in the vapor or liquid phase while contacting the activated porous metalloaluminophosphate molecular sieve. Such contacting can take place under a variety of temperature and pressure conditions, provided the mixture of aldehyde and water is maintained in the vapor or liquid phase. Contacting can take place having a temperature range of about −40° C. to about 150° C., preferably, about −20° C. to about 100° C., more preferably, about −10° C. to about 50° C., most preferably, about 0° C. to about 30° C.

Contacting can take place by immersing or suspending the molecular sieve or catalyst in the aldehyde and water mixture in a vapor and/or liquid phase. Contacting of the molecular sieve or catalyst with the water and aldehyde mixture is conveniently performed by storing the activated porous molecular sieve or catalyst in a vessel containing the vapor and/or liquid mixture of aldehyde and water having the desired aldehyde content.

After contacting with the vapor and/or liquid mixture of aldehyde and water, preferably, at least 80% of the pore volume of the activated porous molecular sieve has been filled with the mixture of aldehyde and water, more preferably, at least 85% of the pore volume of the activated porous molecular sieve has been filled with the mixture of aldehyde and water, more preferably, at least 90% of the pore volume of the activated porous molecular sieve has been filled with the mixture of aldehyde and water, most preferably, at least 95% of the pore volume of the activated porous molecular sieve has been filled with the mixture of aldehyde and water.

The aldehyde in the pore of the porous molecular sieve can be used to form an integrated hydrocarbon co-catalyst within the pore structure of the molecular sieve. The integrated hydrocarbon co-catalyst is preferably a single ring aromatic compound. More preferably, the integrated hydrocarbon co-catalyst is a benzene-based compound. Still more preferably, the integrated hydrocarbon co-catalyst is identified by Solid State Nuclear Magnetic Resonance (SSNMR) spectra comprising a peak in the 18 ppm to 40 ppm region and a peak in the 120 ppm to 150 ppm region. Alternatively, the intensity of the peak in the 18 ppm to 40 ppm region is negligible, with a single peak near 128 ppm. The integrated hydrocarbon co-catalyst can be formed by slowly heating the activated porous molecular sieve under condition suitable to form integrated hydrocarbon co-catalyst as disclosed in U.S. application Ser. No. 10/712,952 which is herein fully incorporated by reference. Alternatively, the integrated hydrocarbon co-catalyst can be formed by direct using the stored molecular sieve without removing the aldehyde absorbed in the pore. An important advantage of using the aldehyde pretreated molecular sieve is that a substantial increase in selectivity to ethylene and propylene content in the olefin product when the molecular sieve is used to produce light olefins by contacting the molecular sieve with oxygenate in the oxygenate reaction zone under suitable condition.

### 3. Molecular Sieves and Catalysts Thereof

The metalloaluminophosphate molecular sieves, which may be used in the present invention, have been described in detail in numerous publications including for example, U.S. Pat. No. 4,567,029 (MeAPO where Me is Mg, Mn, Zn, or Co), U.S. Pat. No. 4,440,871 (SAPO), European Patent Application EP-A-0 159 624 (ELAPSO where E1 is As, Be, B, Cr, Co, Ga, Ge, Fe, Li, Mg, Mn, Ti or Zn), U.S. Pat. No. 4,554,143 (FeAPO), U.S. Pat. Nos. 4,822,478, 4,683,217, 4,744,885 (FeAPSO), EP-A-0 158 975 and U.S. Pat. No. 4,935,216 (ZnAPSO, EP-A-0 161 489 (CoAPSO), EP-A-0 158 976 (ELAPO, where EL is Co, Fe, Mg, Mn, Ti or Zn), U.S. Pat. No. 4,310,440 (AlPO4), EP-A-0 158 350 (SENAPSO), U.S. Pat. No. 4,973,460 (LiAPSO), U.S. Pat. No. 4,789,535 (LiAPO), U.S. Pat. No. 4,992,250 (GeAPSO), U.S. Pat. No. 4,888,167 (GeAPO), U.S. Pat. No. 5,057,295 (BAPSO), U.S. Pat. No. 4,738,837 (CrAPSO), U.S. Pat. Nos. 4,759,919, and 4,851,106 (CrAPO), U.S. Pat. Nos. 4,758,419, 4,882,038, 5,434,326 and 5,478,787 (MgAPSO), U.S. Pat. No. 4,554,143 (FeAPO), U.S. Pat. No. 4,894,213 (AsAPSO), U.S. Pat. No. 4,913,888 (AsAPO), U.S. Pat. Nos. 4,686,092, 4,846,956 and 4,793,833 (MnAPSO), U.S. Pat. Nos. 5,345,011 and 6,156,931 (MnAPO), U.S. Pat. No. 4,737,353 (BeAPSO), U.S. Pat. No. 4,940,570 (BeAPO), U.S. Pat. Nos. 4,801,309, 4,684,617 and 4,880,520 (TiAPSO), U.S. Pat. Nos. 4,500,651, 4,551,236 and 4,605,492 (TiAPO), U.S. Pat. No. 4,824,554, 4,744,970 (CoAPSO), U.S. Pat. No. 4,735,806 (GaAPSO) EP-A-0 293 937 (QAPSO, where Q is framework oxide unit [QO2]), as well as U.S. Pat. Nos. 4,567,029, 4,686,093, 4,781,814, 4,793,984, 4,801,364, 4,853,197, 4,917,876, 4,952,384, 4,956,164, 4,956,165, 4,973,785, 5,241,093, 5,493,066 and 5,675,050, all of which are herein fully incorporated by reference.

Other metalloaluminophosphate molecular sieves include those described in EP-0 888 187 B1 (microporous crystalline metalloaluminophosphates (UIO-6)), U.S. Pat. No. 6,004,898 (molecular sieve and an alkaline earth metal), U.S. patent application Ser. No. 09/511,943 filed Feb. 24, 2000 (integrated hydrocarbon co-catalyst), PCT WO 01/64340 published Sep. 7, 2001 (thorium containing molecular sieve), and R. Szostak, Handbook of Molecular Sieves, Van Nostrand Reinhold, New York, N.Y. (1992), which are all herein fully incorporated by reference.

The preferred molecular sieves are SAPO molecular sieves, and metal substituted SAPO molecular sieves. In one embodiment, the metal is an alkali metal of Group IA of the Periodic Table of Elements, an alkaline earth metal of Group IIA of the Periodic Table of Elements, a rare earth metal of Group IIIB, including the Lanthanides: lanthanum, cerium, praseodymium, neodymium, samarium, europium, gadolinium, terbium, dysprosium, holmium, erbium, thulium, ytterbium and lutetium; and scandium or yttrium of the Periodic Table of Elements, a transition metal of Groups IVB, VB, VIB, VIIB, VIIIB, and IB of the Periodic Table of Elements, or mixtures of any of these metal species. In one preferred embodiment, the metal is selected from the group consisting of Co, Cr, Cu, Fe, Ga, Ge, Mg, Mn, Ni, Sn, Ti, Zn and Zr, and mixtures thereof.

The metalloaluminophosphate molecular sieve may be represented by the empirical formula, on an anhydrous basis:
mR:(MₓAl_{y}P_{z})O₂
wherein R represents at least one templating agent, preferably an organic templating agent; m is the number of moles of R per mole of (MₓAl_{y}P_{z})O₂ and m has a value from 0 to 1, preferably 0 to 0.5, and most preferably from 0 to 0.3; x, y, and z represent the mole fraction of M, Al and P as tetrahedral oxides, where M is a metal selected from one of Group IA, IIA, IB, IIIB, IVB, VB, VIB, VIIB, VIIIB and Lanthanide's of the Periodic Table of Elements, preferably M is selected from one of the group consisting of Co, Cr, Cu, Fe, Ga, Ge, Mg, Mn, Ni, Sn, Ti, Zn and Zr. In an embodiment, m is greater than or equal to 0.2, and x, y and z are greater than or equal to 0.01. In another embodiment, m is greater than 0.1 to about 1, x is greater than 0 to about 0.25, y is in the range of from 0.4 to 0.5, and z is in the range of from 0.25 to 0.5, more preferably m is from 0.15 to 0.7, x is from 0.01 to 0.2, y is from 0.4 to 0.5, and z is from 0.3 to 0.5.

Non-limiting examples of SAPO and ALPO molecular sieves of the invention include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ALPO-5, ALPO-11, ALPO-18, ALPO-31, ALPO-34, ALPO-36, ALPO-37, ALPO-46, and metal containing molecular sieves thereof. The more preferred molecular sieves include one or a combination of SAPO-18, SAPO-34, SAPO-35, SAPO-44, SAPO-56, ALPO-18 and ALPO-34, even more preferably one or a combination of SAPO-18, SAPO-34, ALPO-34 and ALPO-18, and metal containing molecular sieves thereof, and most preferably one or a combination of SAPO-34 and ALPO-18, and metal containing molecular sieves thereof.

As used herein, the term mixture is synonymous with combination and is considered a composition of matter having two or more components in varying proportions, regardless of their physical state. In particular, it encompasses physical mixtures as well as intergrowths of at least two different molecular sieve structures; such as for example those described in PCT Publication No. WO 98/15496 and co-pending U.S. Ser. No. 09/924,016 filed Aug. 7, 2001. In an embodiment, the molecular sieve is an intergrowth material having two or more distinct phases of crystalline structures within one molecular sieve composition. In another embodiment, the molecular sieve comprises at least one intergrown phase of AEI and CHA framework-types. For example, SAPO-18, ALPO-18 and RUW-18 have an AEI framework-type, and SAPO-34 has a CHA framework-type. In a further embodiment the molecular sieve comprises a mixture of intergrown material and non-intergrown material.

As used herein, the term maintaining catalytic activity means preserving the catalytic activity of the molecular sieve of interest against deactivation over time. The catalytic activities of a molecular sieve is maintained if the catalytic activities of a molecular sieve stored with contacting the agent of this invention for a certain period of time is equal or higher than the catalytic activity of a molecular sieve stored in contacting with atmosphere for the same amount of time.

The method of maintaining the catalytic activity of an activated porous molecular sieve of the present invention may be utilized with metalloaluminophosphate molecular sieves which are particularly unstable to moisture exposure e.g., morpholine templated SAPO-34 and may also be used to maintain relatively moisture insensitive molecular sieves such as dual templated (DPA and TEAOH) SAPO-34 materials which may be significantly affected during extended periods of storing or on exposure to water vapor.

Generally, metalloaluminophosphate molecular sieves are synthesized by the hydrothermal crystallization of one or more of a source of aluminum, a source of phosphorous, a source of silicon, a templating agent, and a metal containing compound. Typically, a combination of sources of silicon, aluminum and phosphorous, optionally with one or more templating agents and/or one or more metal containing compounds are placed in a sealed pressure vessel, optionally lined with an inert plastic such as polytetrafluoroethylene, and heated, under a crystallization pressure and temperature, until a crystalline material is formed, and then recovered by filtration, centrifugation and/or decanting. Examples of metalloaluminophosphate molecular sieve synthesis conditions have been described in U.S. Pat. Nos. 4,440,871, 4,861,743, 5,096,684, and 5,126,308, which are all herein fully incorporated by reference.

Non-limiting examples of templating agents-include, cyclohexylamine, morpholine, di-n-propylamine (DPA), tripropylamine, triethylamine (TEA), triethanolamine, piperidine, cyclohexylamine, 2-methylpyridine, N,N-dimethylbenzylamine, N,N-diethylethanolamine, dicyclohexylamine, N,N-dimethylethanolamine, choline, N,N′-dimethylpiperazine, 1,4-diazabicyclo(2,2,2)octane, N′,N′,N,N-tetramethyl-(1,6)hexanediamine, N-methyldiethanolamine, N-methyl-ethanolamine, N-methyl piperidine, 3-methyl-piperidine, N-methylcyclohexylamine, 3-methylpyridine, 4-methyl-pyridine, quinuclidine, N,N′-dimethyl-1,4-diazabicyclo(2,2,2)octane ion; di-n-butylamine, neopentylamine, di-n-pentylamine, isopropylamine, t-butyl-amine, ethylenediamine, pyrrolidine, and 2-imidazolidone. The preferred templating agent or template is a tetraethylammonium compound, such as tetraethyl ammonium hydroxide (TEAOH), tetraethyl ammonium phosphate, tetraethyl ammonium fluoride, tetraethyl ammonium bromide, tetraethyl ammonium chloride and tetraethyl ammonium acetate. In one embodiment, a combination of two or more of any of the above templating agents is used in combination with one or more of a silicon-, aluminum-, and phosphorous-source.

Other suitable metalloaluminophosphate molecular sieves for use in the present invention may be prepared as described in U.S. Pat. No. 5,879,655 (controlling the ratio of the templating agent to phosphorous), U.S. Pat. No. 6,005,155 (use of a modifier without a salt), U.S. Pat. No. 5,475,182 (acid extraction), U.S. Pat. No. 5,962,762 (treatment with transition metal), U.S. Pat. Nos. 5,925,586 and 6,153,552 (phosphorous modified), U.S. Pat. No. 5,925,800 (monolith supported), U.S. Pat. No. 5,932,512 (fluorine treated), U.S. Pat. No. 6,046,373 (electromagnetic wave treated or modified), U.S. Pat. No. 6,051,746 (polynuclear aromatic modifier), U.S. Pat. No. 6,225,254 (heating template), PCT WO 01/36329 published May 25, 2001 (surfactant synthesis), PCT WO 01/25151 published Apr. 12, 2001 (staged acid addition), PCT WO 01/60746 published Aug. 23, 2001 (silicon oil), U.S. patent application Ser. No. 09/929,949 filed Aug. 15, 2001 (cooling molecular sieve), U.S. patent application Ser. No. 09/615,526 filed Jul. 13, 2000 (metal impregnation including copper), U.S. patent application Ser. No. 09/672,469 filed Sep. 28, 2000 (conductive microfilter), and U.S. patent application Ser. No. 09/754,812 filed Jan. 4, 2001(freeze drying the molecular sieve), which are all herein fully incorporated by reference.

In one embodiment, the metalloaluminosphosphate molecular sieve is activated after molecular sieve crystallization. For this purpose, the templating agent is substantially, preferably completely, removed after crystallization by numerous techniques well known to a skilled artisan, for example, heat treatments such as calcination. Calcination involves contacting the molecular sieve containing the templating agent with a gas, preferably containing oxygen, at any desired concentration at an elevated temperature sufficient to either partially or completely decompose and oxidize the templating agent.

In one embodiment, the molecular sieve has a Si/Al2 ratio less than 0.65, preferably less than 0.40, more preferably less than 0.32, and most preferably less than 0.20.

Once the molecular sieve is synthesized the molecular sieve may then be handled or stored by contacting the molecular sieve with a gas or liquid water/aldehyde mixture to prevent water degradation of the molecular sieve. The protected molecular sieve can then be formulated into a molecular sieve catalyst composition. In another embodiment, the metalloaluminophosphate molecular sieve as synthesized, i.e. without having removed the template (for example in the form of a wet filter cake), may be formulated into a catalyst composition. Activation of the molecular sieve then occurs from within the catalyst composition, once the catalyst composition is heated under conditions that allow partial or complete removal of the template from the molecular sieve pore structure.

Molecular sieve activation thus can occur prior to or after the molecular sieve has been formulated into a catalyst composition. In each of these embodiments, the catalytic sites of the molecular sieve can be protected against water degradation during molecular sieve and/or catalyst handling and/or storage by maintaining the molecular sieve or the catalyst in contact with a vapor and/or liquid mixture of water and aldehyde.

In either instance a catalyst composition can be made by combining the metalloaluminophosphate molecular sieve with a binder and/or a matrix material to form a molecular sieve catalyst composition. This catalyst composition is formed into useful shaped and sized particles by well-known techniques such as spray drying, pelletizing, extrusion, and the like. If the molecular sieve has been activated before combining with the binder and/or the matrix material, it is best to handle and store the molecular sieve in contact with a vapor and/or liquid mixture of water and aldehyde according to the present invention, for as long as practically possible. The water and aldehyde mixture used as protecting agent can be, but does not necessarily need to be, removed before the molecular sieve is combined with the binder and/or matrix material. The water and aldehyde mixtures used according to the present invention are suitable mediums for formulating molecular sieves into catalysts. In an embodiment, the activated porous metalloaluminophosphate molecular sieves can be stored as slurries in the aldehyde and water mixtures of the invention after molecular sieve synthesis. The other catalyst formulation agents can then be added to these water and aldehyde molecular sieve slurries at the time of catalyst formulation.

There are many different binders that are useful in forming the molecular sieve catalyst composition. Non-limiting examples of binders that are useful alone or in combination include various types of hydrated alumina, silicas, and/or other inorganic oxide sol. One preferred alumina containing sol is aluminum chlorhydrol. The inorganic oxide sol acts like glue binding the synthesized molecular sieves and other materials such as the matrix together, particularly after thermal treatment. Upon heating, the inorganic oxide sol, preferably having a low viscosity, is converted into an inorganic oxide matrix component. For example, an alumina sol will convert to an aluminum oxide matrix following heat treatment.

Aluminum chlorhydrol, a hydroxylated aluminum based sol containing a chloride counter ion, has the general formula of AlₘOₙ(OH)ₒClₚ.x(H₂O) wherein m is 1 to 20, n is 1 to 8, o is 5 to 40, p is 2 to 15, and x is 0 to 30. In one embodiment, the binder is Al₁₃O₄(OH)₂₄Cl₇.12(H₂O) as is described in G. M. Wolterman, et al., Stud. Surf Sci. and Catal., 76, pages 105-144 (1993), which is herein incorporated by reference. In another embodiment, one or more binders are combined with one or more other non-limiting examples of alumina materials such as aluminum oxyhydroxide, γ-alumina, boehmite, diaspore, and transitional aluminas such as α-alumina, β-alumina, γ-alumina, δ-alumina, ε-alumina, κ-alumina, and ρ-alumina, aluminum trihydroxide, such as gibbsite, bayerite, nordstrandite, doyelite, and mixtures thereof.

In another embodiment, the binders are alumina sols, predominantly comprising aluminum oxide, optionally including some silicon. In yet another embodiment, the binders are peptized alumina made by treating alumina hydrates such as pseudobohemite, with an acid, preferably an acid that does not contain a halogen, to prepare sols or aluminum ion solutions. Non-limiting examples of commercially available colloidal alumina sols include Nalco 8676 available from Nalco Chemical Co., Naperville, Ill., and Nyacol® available from The PQ Corporation, Valley Forge, Pa.

The metalloaluminophosphate molecular sieve may be combined with one or more matrix material(s). Matrix materials are typically effective in reducing overall catalyst cost, act as thermal sinks assisting in shielding heat from the catalyst composition for example during regeneration, densifying the catalyst composition, increasing catalyst strength such as crush strength and attrition resistance, and to control the rate of conversion in a particular process.

Non-limiting examples of matrix materials include one or more of: rare earth metals, metal oxides including titania, zirconia, magnesia, thoria, beryllia, quartz, silica or sols, and mixtures thereof, for example silica-magnesia, silica-zirconia, silica-titania, silica-alumina and silica-alumina-thoria. In an embodiment, matrix materials are natural clays such as those from the families of montmorillonite and kaolin. These natural clays include sabbentonites and those kaolins known as, for example, Dixie, McNamee, Georgia and Florida clays. Non-limiting examples of other matrix materials include: haloysite, kaolinite, dickite, nacrite, or anauxite. In one embodiment, the matrix material, preferably any of the clays, are subjected to well known modification processes such as calcination and/or acid treatment and/or chemical treatment.

In one preferred embodiment, the matrix material is a clay or a clay-type composition, preferably the clay or clay-type composition having a low iron or titania content, and most preferably the matrix material is kaolin. Kaolin has been found to form a pumpable, high solid content slurry; it has a low fresh surface area, and it packs together easily due to its platelet structure. A preferred average particle size of the matrix material, most preferably kaolin, is from about 0.1 μm to about 0.6 μm with a D90 particle size distribution of less than about 1 μm.

In one embodiment, the binder, the molecular sieve and the matrix material are combined in the presence of a liquid to form a molecular sieve catalyst composition, where the amount of binder is from about 2% by weight to about 30% by weight, preferably from about 5% by weight to about 20% by weight, and more preferably from about 7% by weight to about 15% by weight, based on the total weight of the binder, the molecular sieve and matrix material, excluding the liquid (after calcination).

In another embodiment, the weight ratio of the binder to the matrix material used in the formation of the molecular sieve catalyst composition is from 0:1 to 1:15, preferably 1:15 to 1:5, more preferably 1:10 to 1:4, and most preferably 1:6 to 1:5. It has been found that a higher sieve content, lower matrix content, increases the molecular sieve catalyst composition performance, however, lower sieve content, higher matrix material, improves the attrition resistance of the composition.

Upon combining the molecular sieve and the matrix material, optionally with a binder, in a liquid to form a slurry, mixing, preferably rigorous mixing is needed to produce a substantially homogeneous mixture containing the molecular sieve. Non-limiting examples of suitable liquids include one or a combination of water, alcohol, ketones, aldehydes, and/or esters. The most preferred liquids are water/alcohol mixtures and water. In one embodiment, the slurry is colloid-milled for a period of time sufficient to produce the desired slurry texture, sub-particle size, and/or sub-particle size distribution. In the present invention the use of a mixture of aldehyde and water is beneficial in the catalyst formulation process as the water/aldehyde mixture protects the molecular sieve from degradation during the formulation process.

The molecular sieve and matrix material, and the optional binder, may be in the same or different liquid, and may be combined in any order, together, simultaneously, sequentially, or a combination thereof. In one embodiment, the slurry of the molecular sieve, binder and matrix materials is mixed or milled to achieve a sufficiently uniform slurry of sub-particles of the molecular sieve catalyst composition that is then fed to a forming unit that produces the molecular sieve catalyst composition. In a preferred embodiment, the forming unit is a spray dryer. Typically, the forming unit is maintained at a temperature sufficient to remove most of the liquid from the slurry, and from the resulting molecular sieve catalyst composition. The resulting catalyst composition when formed in this way takes the form of microspheres.

In another embodiment, the formulated molecular sieve catalyst composition contains from about 1% to about 99%, more preferably from about 5% to about 90%, and most preferably from about 10% to about 80%, by weight of the molecular sieve based on the total weight of the molecular sieve catalyst composition.

In another embodiment, the weight percent of binder in or on the spray dried molecular sieve catalyst composition based on the total weight of the binder, molecular sieve, and matrix material is from about 2% by weight to about 30% by weight, preferably from about 5% by weight to about 20% by weight, and more preferably from about 7% by weight to about 15% by weight.

Once the molecular sieve catalyst composition is formed in a substantially dry or dried state, to further harden and/or activate the formed catalyst composition, a heat treatment such as calcination, at an elevated temperature is usually performed. A conventional calcination environment is air that typically includes a small amount of water vapor. Typical calcination temperatures are in the range from about 400° C. to about 1,000° C., preferably from about 500° C. to about 800° C., and most preferably from about 550° C. to about 700° C., preferably in a calcination environment such as air, nitrogen, helium, flue gas (combustion product lean in oxygen), or any combination thereof.

In one embodiment, calcination of the formulated molecular sieve catalyst composition is carried out in any number of well known devices including rotary calciners, fluid bed calciners, batch ovens, and the like. Calcination time is typically dependent on the degree of hardening of the molecular sieve catalyst composition and the temperature.

In a preferred embodiment, the molecular sieve catalyst composition is heated in nitrogen at a temperature of from about 600° C. to about 700° C. Heating is carried out for a period of time typically from 30 minutes to 15 hours, preferably from 1 hour to about 10 hours, more preferably from about 1 hour to about 5 hours, and most preferably from about 2 hours to about 4 hours.

Other methods for activating a molecular sieve catalyst composition, are described in, for example, U.S. Pat. No. 5,185,310 (heating molecular sieve of gel alumina and water to 450 C), PCT WO 00/75072 published Dec. 14, 2000 (heating to leave an amount of template), and U.S. application Ser. No. 09/558,774 filed Apr. 26, 2000 and published as PCT Publication No. WO 01/80995 to Janssen et. al (rejuvenation of molecular sieve), which are all herein fully incorporated by reference.

In addition to the metalloaluminophosphate molecular sieve, the catalyst compositions of the present invention may comprise one or several additional catalytically active materials. In one embodiment, one or several metalloaluminophosphate molecular sieves are combined with one more of the following non-limiting examples of catalytically active molecular sieves described in the following: Beta (U.S. Pat. No. 3,308,069), ZSM-5 (U.S. Pat. Nos. 3,702,886, 4,797,267 and 5,783,321), ZSM-11 (U.S. Pat. No. 3,709,979), ZSM-12 (U.S. Pat. No. 3,832,449), ZSM-12 and ZSM-38 (U.S. Pat. No. 3,948,758), ZSM-22 (U.S. Pat. No. 5,336,478), ZSM-23 (U.S. Pat. No. 4,076,842), ZSM-34 (U.S. Pat. No. 4,086,186), ZSM-35 (U.S. Pat. No. 4,016,245, ZSM-48 (U.S. Pat. No. 4,397,827), ZSM-58 (U.S. Pat. No. 4,698,217), MCM-1 (U.S. Pat. No. 4,639,358), MCM-2 (U.S. Pat. No. 4,673,559), MCM-3 (U.S. Pat. No. 4,632,811), MCM-4 (U.S. Pat. No. 4,664,897), MCM-5 (U.S. Pat. No. 4,639,357), MCM-9 (U.S. Pat. No. 4,880,611), MCM-10 (U.S. Pat. No. 4,623,527)MCM-14 (U.S. Pat. No. 4,619,818), MCM-22 (U.S. Pat. No. 4,954,325), MCM-41 (U.S. Pat. No. 5,098,684), M-41S (U.S. Pat. No. 5,102,643), MCM-48 (U.S. Pat. No. 5,198,203), MCM-49 (U.S. Pat. No. 5,236,575), MCM-56 (U.S. Pat. No. 5,362,697), ALPO-11 (U.S. Pat. No. 4,310,440), titanium aluminosilicates (TASO), TASO-45 (EP-A-0 229,-295), boron silicates (U.S. Pat. No. 4,254,297), titanium aluminophosphates (TAPO) (U.S. Pat. No. 4,500,651), mixtures of ZSM-5 and ZSM-11 (U.S. Pat. No. 4,229,424), ECR-18 (U.S. Pat. No. 5,278,345).

In another embodiment, the metalloaluminophosphate may be bound to another molecular sieve, as disclosed for example in the following: SAPO-34 bound ALPO-5 (U.S. Pat. No. 5,972,203), PCT WO 98/57743 published Dec. 23, 1988 (molecular sieve and Fischer-Tropsch), U.S. Pat. No. 6,300,535 (MFI-bound zeolites), and mesoporous molecular sieves (U.S. Pat. Nos. 6,284,696, 5,098,684, 5,102,643 and 5,108,725), which are all herein fully incorporated by reference. Binder may no longer be necessary in such systems.

In a further embodiment, the metalloaluminophosphate molecular sieve may be combined with a metal catalyst, for example as a Fischer-Tropsch catalyst.

The catalyst compositions of the present invention may comprise one or more metalloaluminophosphate molecular sieves, which may be combined with one or more non-metalloaluminophosphate molecular sieves such as zeolites of zeolite-like molecular sieves described above. It is preferred that the catalyst comprises metalloaluminophosphates as the sole molecular sieve component.

### 4. Maintaining Conditions

In the present invention an “active porous molecular sieve” encompasses any of the following: a) an activated porous molecular sieve; or, b) an activated porous molecular sieve in a formulated catalyst particle, which has been stored for an extended period of time after synthesis; or, c) a porous molecular sieve material, which has been used in a catalytic process and has been removed from that process or temporarily retained under non-optimum process conditions such as in a shutdown phase. Similarly, in the context of the invention, an aged catalyst composition is an activated catalyst composition as formed, which has been stored for an extended period of time after synthesis, or it is a used catalyst composition, which has been used in a catalytic process and has been removed from that process or temporarily retained under non-optimum process conditions such as in a shutdown phase. By extended periods of time is meant a period greater than one hour, preferably greater than 6 hours, preferably greater than 12 hours, more preferably more than 24 hours, even more preferably greater than 36 hours, yet even more preferably greater than 48 hours, and most preferably greater than 72 hours. Preferably, the period of storage in the presence of a water and aldehyde mixture does not extend beyond 12 months. The period of maintaining while in contact with the water and aldehyde mixture may be under a variety of temperature and pressure conditions, provided the aldehyde/water mixture is in the gas or liquid states and the activated porous molecular sieve has its pore volume filled with the mixture of water and aldehyde by at least 80%, more preferably by at least 85%, even more preferably by at least 90%, and most preferably by at least 95%.

In addition to contacting the molecular sieve or catalyst with a mixture of water and aldehyde, storage can be undertaken under an inert atmosphere, for example in a sealed container such as storage drum or holding facility after manufacture of the sieve or catalyst.

Without being bound by any particular theory, it is believed that when the aldehyde has a kinetic diameter less than the diameter of the pores of the porous molecular sieve, which allows the aldehyde to enter the cage of the molecular sieve, then the water/aldehyde is able to occupy a portion of the pores within the porous molecular sieve, and in doing so protects them from attack by moisture during storage and handling. In one embodiment of the present invention, at least 80%, more preferably 85%, even more preferable 90%, and most preferably 95%, of pore volume of porous molecular sieve is occupied by the mixture of water and aldehyde.

When the activated porous molecular sieve is maintained in contact with a vapor and/or liquid mixture of aldehyde and water of the present invention, the molecular sieve or molecular sieve-containing catalyst composition is stable. By stable is meant that there is less reduction in the catalytic activity of the molecular sieve maintained in contact with the water and aldehyde mixture compared to the same molecular sieve maintained under the same conditions, but without contact with a water and aldehyde mixture of the invention. The molecular sieve or catalyst may be maintained in contact with the aldehyde/water mixture for an extended period of time, which is typically at least 6 hours, preferably at least 12 hours and which may be for any period of storage, shipping or handling greater than 12 hours.

In one embodiment of the present invention, the molecular sieve is contacted with a water/acetyl aldehyde mixture at ambient conditions. Ideally the molecular sieve or catalyst is held in this state as long as possible before use, provided the methanol uptake index, as defined in this application, of the molecular sieve or catalyst has not dropped below 0.5.

In the present invention, contacting the molecular sieve with a mixture of aldehyde and water is effective in retaining the methanol uptake properties of the molecular sieve. The molecular sieve stored in contacting with a mixture of aldehyde and water has higher MUI than the molecular sieve stored without contacting a mixture of aldehyde and water for same period of time.

One way to express the loss of catalytic activity over time, is to calculate the methanol uptake index (MUI) of molecular sieve or catalyst. The MUI is defined as the ratio between the maximum methanol uptake capacity (wt %) of an activated porous molecular sieve or catalyst (i.e., the initial methanol uptake capacity) and the methanol uptake capacity (wt %) of the activated porous molecular sieve or catalyst after the molecular sieve or catalyst has been stored for a given period of time. The MUI is the weight percent increase measured by flowing a methanol containing nitrogen gas with a methanol partial pressure of 9.12 KPa-a (0.09 ATM) for 180 minutes at 30° C. Conveniently, a thermogravimetric analyzer (TGA) may be used for measuring MUI by following the following steps: a) placing a x gram of freshly dried catalyst (in air at 650° C. for 40 minutes) in the TGA, b) contacting the catalyst with a methanol containing nitrogen gas with a methanol partial pressure of 9.12 KPa-a (0.09 ATM) for 180 minutes at 30° C., c) measuring the weight increase (y) of the catalyst after step (b), and d) the MUI is calculated as y/x. According to this invention, it is preferred that the MUI be at least 0.15, preferably at least 0.4, more preferably at least 0.45 and most preferably at least 0.5, at the time of catalytic contact. Contact with the feed should thus occur before the MUI drops below 0.15.

In the context of the present invention aged metalloaluminophosphates are typically present in large amounts i.e. the bulk state. By bulk state is meant in the form of a large batch of material or catalyst comprising the metalloaluminophosphate. Typically a bulk sample has a batch size of greater than 1 kilogram, preferably greater than 10 kilograms, most preferably greater than 50 kilograms. Storage may be undertaken in the presence of an inert gas in addition to the water and aldehyde mixture. The present invention makes its possible to utilize grades of inert gases which were hitherto unacceptable for metalloaluminophosphate molecular sieve storage due to their moisture content. Such gases may be of lower purity and quality e.g. they may contain higher than normal levels of impurities such as oxygen and/or moisture.

In another embodiment, water and aldehyde mixtures of the invention can be used to protect catalyst from loss of catalytic activity during reactor operation interruptions that require temporary catalyst storage. During conversion processes, it may be necessary to shut the reactor down in either an emergency or in a planned shutdown and maintenance cycle. When this occurs it is often necessary to remove the used catalyst from the reactor and to place the catalyst into temporary storage, which is usually under an inert atmosphere. Sometimes removal is not necessary or desirable and the catalyst is maintained within the plant itself. In both situations the catalyst is at risk of losing its catalytic activity and/or other properties due to aging effects. During such events catalyst can be effectively protected against the effects of water by blanketing with mixtures of aldehyde and water. In this embodiment the used catalyst may be treated with a vapor and/or liquid mixture of aldehyde and water as it is removed from the plant; the water/aldehyde mixture can be maintained in contact with the catalyst as it is re-introduced to the plant. In an alternative embodiment the used catalyst is treated within the plant during or after shutdown.

In another embodiment, water and aldehyde mixtures of the invention can be used to generate co-catalyst within the pore structure of the molecular sieve. The co-catalyst can be formed by slowly heating the activated porous molecular sieve under condition suitable to form integrated hydrocarbon co-catalyst as disclosed in U.S. patent application Ser. No. 10/712,952, which is herein fully incorporated by reference. The molecular sieve containing the co-catalyst is used to convert oxygenate to an olefin product containing a significant amount of ethylene and propylene, with relatively low amount of undesired by-products.

After the storing period is over, it may be desirable to separate the metalloaluminophosphate molecular sieve from contact with the liquid aldehyde and water mixture. This can be achieved by any method, for example, filtration, centrifugation, decanting, drying, calcination, or any combination of these steps.

### 5. Using the Molecular Sieve Catalyst Compositions

The molecular sieve catalysts and compositions of the present invention are useful in a variety of processes including: cracking, hydrocracking, isomerization, polymerization, reforming; hydrogenation, dehydrogenation, dewaxing, hydrodewaxing, absorption, alkylation, transalkylation, dealkylation, hydrodecylization, disproportionation, oligomerization, dehydrocyclization, and combinations thereof.

The preferred processes of the present invention include a process directed to the conversion of a feedstock comprising one or more oxygenates to one or more derivative products, such as methylamines or olefins, most preferably olefin(s).

Prior to being used in these processes, the liquid water and aldehyde mixture used to protect the molecular sieve from ambient degradation can be removed if desired. However, it is desired not to remove the water and aldehyde mixture filled in the pore of the activated porous molecular sieve before loading into the reactor, or even before contacting with the oxygenated feedstock begins.

In a preferred embodiment of the process of the invention, the feedstock comprises one or more oxygenates, more specifically, one or more organic compound(s) containing at least one oxygen atom. In the most preferred embodiment of the process of invention, the oxygenate in the feedstock is one or more aldehyde(s), preferably aliphatic aldehyde(s) where the aliphatic moiety of the aldehyde(s) has from 1 to 20 carbon atoms, preferably from 1 to 10 carbon atoms, and most preferably from 1 to 4 carbon atoms. The aldehydes useful as feedstock in the process of the invention include lower straight and branched chain aliphatic aldehydes and their unsaturated counterparts.

Non-limiting examples of oxygenates include methanol, ethanol, n-propanol, isopropanol, methyl ethyl ether, dimethyl ether, diethyl ether, di-isopropyl ether, formaldehyde, dimethyl carbonate, dimethyl ketone, acetic acid, and mixtures thereof.

In the most preferred embodiment, the feedstock is selected from one or more of methanol, ethanol, dimethyl ether, diethyl ether or a combination thereof, more preferably methanol and dimethyl ether, and most preferably methanol.

The protected metalloaluminophosphate molecular sieves of the present invention are suitable for use as catalysts in a variety of hydrocarbon conversion processes, including aromatic alkylation, the manufacture of methylamines and the manufacture of olefins. For aromatic alkylation, the feedstock comprises at least one oxygenated hydrocarbon and at least one aromatic hydrocarbon; for methylamine manufacture, the feedstock comprises at least one oxygenated hydrocarbon and ammonia; for the manufacture of olefin(s), the feedstock comprises at least one oxygenated hydrocarbon.

In a preferred embodiment, the feedstock, preferably of one or more oxygenates, is converted in the presence of a molecular sieve catalyst composition into olefin(s) having 2 to 6 carbons atoms, preferably 2 to 4 carbon atoms. More preferably, the olefin(s), alone or combination, are converted from a feedstock containing an oxygenate, preferably an alcohol, most preferably methanol, to the preferred olefin(s) ethylene and/or propylene.

The most preferred process is generally referred to as gas-to-olefins (GTO) or alternatively, methanol-to-olefins (MTO). In a MTO process, typically an oxygenated feedstock, most preferably a methanol containing feedstock, is converted in the presence of a molecular sieve catalyst composition into one or more olefin(s), preferably and predominantly, ethylene and/or propylene, often referred to as light olefin(s).

Prime olefin selectivity wt. % (POS), being the sum of weight percent of ethylene and propylene in the product, can be calculated from the measured yields and conversions at certain cumulative methanol converted per gram of molecular sieve (CMCPS). In one embodiment of this invention, the GTO hydrocarbon product of the activated porous molecular sieve being stored in contact with the agent has at least 0.1 wt. %, preferably, at least 1 wt. %, even preferably, at least 3 wt. % higher, most preferably, at least 5 wt. % higher primary olefin selectivity (POS) as compared to the same activated porous molecular sieve without being stored in contact with the agent.

The feedstock can contain one or more diluent(s), typically used to reduce the concentration of the feedstock, and are generally non-reactive to the feedstock or molecular sieve catalyst composition. Non-limiting examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred. The diluent, water, is used either in a liquid or a vapor form, or a combination thereof. The diluent is either added directly to a feedstock entering into a reactor or added directly into a reactor, or added with a molecular sieve catalyst composition.

The process for converting a feedstock, especially a feedstock containing one or more oxygenates, in the presence of a molecular sieve catalyst composition of the invention, is carried out in a reaction process in a reactor, where the process is a fixed bed process, a fluidized bed process (includes a turbulent bed process), preferably a continuous fluidized bed process, and most preferably a continuous high velocity fluidized bed process.

The reaction processes can take place in a variety of catalytic reactors such as hybrid reactors that have a dense bed or fixed bed reaction zones and/or fast fluidized bed reaction zones coupled together, circulating fluidized bed reactors, riser reactors, and the like. Suitable conventional reactor types are described in for example U.S. Pat. No. 4,076,796, U.S. Pat. No. 6,287,522 (dual riser), and Fluidization Engineering, D. Kunii and O. Levenspiel, Robert E. Krieger Publishing Company, New York, N.Y. 1977, which are all herein fully incorporated by reference.

The preferred reactor type are riser reactors generally described in Riser Reactor, Fluidization and Fluid-Particle Systems, pages 48 to 59, F. A. Zenz and D. F. Othmo, Reinhold Publishing Corporation, New York, 1960, and U.S. Pat. No. 6,166,282 (fast-fluidized bed reactor), and U.S. patent application Ser. No. 09/564,613, filed May 4, 2000 (multiple riser reactor), which are all herein fully incorporated by reference.

In the preferred embodiment, a fluidized bed process or high velocity fluidized bed process includes a reactor system, a regeneration system and a recovery system.

The reactor system preferably is a fluid bed reactor system having a first reaction zone within one or more riser reactor(s) and a second reaction zone within at least one disengaging vessel, preferably comprising one or more cyclones. In one embodiment, the one or more riser reactor(s) and disengaging vessel is contained within a single reactor vessel. Fresh feedstock, preferably containing one or more oxygenates, optionally with one or more diluent(s), is fed to the one or more riser reactor(s) in which a molecular sieve catalyst composition or coked version thereof is introduced. In one embodiment, the molecular sieve catalyst composition or coked version thereof is contacted with a liquid or gas, or combination thereof, prior to being introduced to the riser reactor(s), preferably the liquid is water or methanol, and the gas is an inert gas such as nitrogen.

The feedstock entering the reactor system is preferably converted, partially or fully, in the first reactor zone into a gaseous effluent that enters the disengaging vessel along with a coked molecular sieve catalyst composition. In the preferred embodiment, cyclone(s) within the disengaging vessel are designed to separate the molecular sieve catalyst composition, preferably a coked molecular sieve catalyst composition, from the gaseous effluent containing one or more olefin(s) within the disengaging zone. Cyclones are preferred, however, gravity effects within the disengaging vessel will also separate the catalyst compositions from the gaseous effluent. Other methods for separating the catalyst compositions from the gaseous effluent include the use of plates, caps, elbows, and the like.

In one embodiment of the disengaging system, the disengaging system includes a disengaging vessel; typically a lower portion of the disengaging vessel is a stripping zone. In the stripping zone the coked molecular sieve catalyst composition is contacted with a gas, preferably one or a combination of steam, methane, carbon dioxide, carbon monoxide, hydrogen, or an inert gas such as argon, preferably steam, to recover adsorbed hydrocarbons from the coked molecular sieve catalyst composition that is then introduced to the regeneration system. In another embodiment, the stripping zone is in a separate vessel from the disengaging vessel and the gas is passed at a gas hourly superficial velocity (GHSV) of from 1 hr⁻¹ to about 20,000 hr⁻¹ based on the volume of gas to volume of coked molecular sieve catalyst composition, preferably at an elevated temperature from 250° C. to about 750° C., preferably from about 350° C. to 650° C., over the coked molecular sieve catalyst composition.

The conversion temperature employed in the conversion process, specifically within the reactor system, is in the range of from about 200° C. to about 1000° C., preferably from about 250° C. to about 800° C., more preferably from about 250° C. to about 750° C., yet more preferably from about 300° C. to about 650° C., yet even more preferably from about 350° C. to about 600° C. most preferably from about 350° C. to about 550° C.

The conversion pressure employed in the conversion process, specifically within the reactor system, varies over a wide range including autogenous pressure. The conversion pressure is based on the partial pressure of the feedstock exclusive of any diluent therein. Typically the conversion pressure employed in the process is in the range of from about 0.1 KPa-a to about 5 MPa-a, preferably from about 5 KPa-a to about 1 MPa-a, and most preferably from about 20 KPa-a to about 500 KPa-a.

The weight hourly space velocity (WHSV), particularly in a process for converting a feedstock containing one or more oxygenates in the presence of a molecular sieve catalyst composition within a reaction zone, is defined as the total weight of the feedstock excluding any diluents to the reaction zone per hour per weight of molecular sieve in the molecular sieve catalyst composition in the reaction zone. The WHSV is maintained at a level sufficient to keep the catalyst composition in a fluidized state within a reactor.

Typically, the WHSV ranges from about 1 hr⁻¹ to about 5000 hr⁻¹, preferably from about 2 hr⁻¹ to about 3000 hr⁻¹, more preferably from about 5 hr⁻¹ to about 1500 hr⁻¹, and most preferably from about 10 hr⁻¹ to about 1000 hr⁻¹. In one preferred embodiment, the WHSV is greater than 20 hr⁻¹; preferably the WHSV for conversion of a feedstock containing methanol and dimethyl ether is in the range of from about 20 hr⁻¹ to about 300 hr⁻¹.

The superficial gas velocity (SGV) of the feedstock including diluent and reaction products within the reactor system is preferably sufficient to fluidize the molecular sieve catalyst composition within a reaction zone in the reactor. The SGV in the process, particularly within the reactor system, more particularly within the riser reactor(s), is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec. See for example U.S. patent application Ser. No. 09/708,753 filed Nov. 8, 2000, which is herein incorporated by reference.

During catalyst use, the catalyst becomes coked. The coked molecular sieve catalyst composition is withdrawn from the disengaging vessel, preferably by one or more cyclones(s), and introduced to the regeneration system. The regeneration system comprises a regenerator where the coked catalyst composition is contacted with a regeneration medium, preferably a gas containing oxygen, under general regeneration conditions of temperature, pressure and residence time.

In an embodiment, a portion of the coked molecular sieve catalyst composition from the regenerator is returned directly to the one or more riser reactor(s), or indirectly, by pre-contacting with the feedstock, or contacting with fresh molecular sieve catalyst composition, or contacting with a regenerated molecular sieve catalyst composition or a cooled regenerated molecular sieve catalyst composition described below.

The regenerated molecular sieve catalyst composition withdrawn from the regeneration system is combined with a fresh molecular sieve catalyst composition and/or re-circulated molecular sieve catalyst composition and/or feedstock and/or fresh gas or liquids, and returned to the riser reactor(s). In one embodiment, a carrier, such as an inert gas, feedstock vapor, steam or the like, semi-continuously or continuously, facilitates the introduction of the regenerated molecular sieve catalyst composition to the reactor system, preferably to the one or more riser reactor(s).

By controlling the flow of the regenerated molecular sieve catalyst composition or cooled regenerated molecular sieve catalyst composition from the regeneration system to the reactor system, the optimum level of coke on the molecular sieve catalyst composition entering the reactor is maintained. There are many techniques for controlling the flow of a molecular sieve catalyst composition described in Michael Louge, Experimental Techniques, Circulating Fluidized Beds, Grace, Avidan and Knowlton, eds. Blackie, 1997 (336-337), which is herein incorporated by reference.

The gaseous effluent is withdrawn from the disengaging system and is passed through a recovery system. There are many well-known recovery systems, techniques and sequences that are useful in separating olefin(s) and purifying olefin(s) from the gaseous effluent. Recovery systems generally comprise one or more or a combination of a various separation, fractionation and/or distillation towers, columns, splitters, or trains, reaction systems such as ethylbenzene manufacture (U.S. Pat. No. 5,476,978) and other derivative processes such as aldehydes, ketones and ester manufacture (U.S. Pat. No. 5,675,041), and other associated equipment for example various condensers, heat exchangers, refrigeration systems or chill trains, compressors, knock-out drums or pots, pumps, and the like.

In order to provide a better understanding of the present invention including representative advantages thereof, the following examples are offered.

### 6. Examples

CHA/AEI SAPO intergrowth catalyst was prepared according to the procedure disclosed in U.S. application Ser. No. 10/995,870, filed Mar. 1, 2001. The CHA/AEI SAPO intergrowth catalyst was calcined for 5 hours at 650° C. under nitrogen, followed by 3 hours under air at 650° C. to form a “freshly calcined CHA/AEI SAPO intergrowth catalyst”.

A portion of the freshly calcined CHA/AEI SAPO intergrowth catalyst was stored under acetaldehyde (Chemical Grade) containing about 5000 ppm water at 2° C. Another portion of the freshly calcined CHA/AEI SAPO intergrowth catalyst was stored under acetaldehyde containing about 5000 ppm water at 25° C. After storage for a given amount of time, the CHA/AEI SAPO intergrowth catalyst was tested for the MTO (Methanol to Olefins) reaction. The methanol conversion of each sample was then compared with the performance of freshly calcined catalyst, not yet contacted with the acetaldehyde/water mixture (base case).

The MTO reaction (Methanol-to-Olefins) was performed in a stainless steel, fixed bed continuous reactor with methanol as feed. The reaction was carried out at a temperature of 450° C., a pressure of 204.7 KPa-a (15 psig), and a WHSV of 26 hr⁻¹. Reaction products were analyzed with an on-line GC. Methanol conversion was calculated as 100−(wt. % methanol+wt. % DME) left in the product. Prime olefin selectivity wt. % (POS), being the sum of weight percent of ethylene and propylene in the product, was calculated from the measured yields and conversions at certain cumulative methanol converted per gram of sieve (CMCPS).

TABLE 1

| | | Hours of storage with acetaldehyde/water | |
|---|---|---|---|
| | | at 2° C. | |
| | Base case | 6 | 72 |
| POS (wt. %) | 65 | 69 | 70 |
| at CMCPS = 1 | | | |

TABLE 2

| | | Hours of storage with acetaldehyde/water | | |
|---|---|---|---|---|
| | | at 25° C. | | |
| | Base case | 3.75 | 24 | 96 |
| POS (wt. %) | 65 | 68 | 72 | 72 |
| at CMCPS = 1 | | | | |

From the above tables, it is clear that the initial light olefin selectivity of the samples stored in contact with an acetaldehyde/water mixture is significantly higher than the initial light olefin selectivity of the samples tested immediately after calcination (activation) and before any storage with the acetaldehyde/water mixture. An increase of about 6-7 wt. % in prime olefin selectivity can be observed over the base case samples when the activated molecular sieve has been stored while in contact with the acetaldehyde/water mixture.

All patents, patent applications, test procedures, priority documents, articles, publications, manuals, and other documents cited herein are fully incorporated by reference to the extent such disclosure is not inconsistent with this invention and for all jurisdictions in which such incorporation is permitted.

When numerical lower limits and numerical upper limits are listed herein, ranges from any lower limit to any upper limit are contemplated.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.

We claim:

1. A method of maintaining catalytic activity of an activated porous molecular sieve, comprising the step of:
contacting the activated porous molecular sieve with an agent having at least 55 wt. % aldehyde and at least 0.1 wt. % water, wherein the activated porous molecular sieve has at least 80% of its pore volume filled with the agent.

2. The method of claim 1, wherein the agent comprises at least 80 wt. % aldehyde.

3. The method of claim 1, wherein the agent comprises at least 90 wt. % aldehyde.

4. The method of claim 1, wherein the agent comprises at least 1 wt. % water.

5. The method of claim 1, wherein the agent comprises at least 5 wt. % water.

6. The method of claim 1, wherein the agent comprises less than 20 wt. % water.

7. The method of claim 1, wherein the aldehyde is an alkyl aldehyde, with the alkyl group having from 1 to 16 carbon atoms, and mixtures thereof.

8. The method of claim 7, wherein the alkyl group has from 1 to 5 carbon atoms.

9. The method of claim 1, wherein the aldehyde is acetaldehyde.

10. The method of claim 1, wherein the activated porous molecular sieve has at least 90% of its pore volume filled with the agent

11. The method of claim 1, wherein the activated porous molecular sieve has at least 95% of its pore volume filled with the agent.

12. The method of claim 1, wherein contacting the activated porous molecular sieve takes place at a temperature of from about −40° C. to about 150° C.

13. The method of claim 12, wherein contacting the activated porous molecular sieve takes place at a temperature of from about 0° C. to about 30° C.

14. The method of claim 1, wherein the activated porous molecular sieve is an activated porous metalloaluminophosphate molecular sieve.

15. The method of claim 14, wherein the activated porous metalloaluminophosphate molecular sieve is selected from SAPO-11, SAPO-17, SAPO-18, SAPO-34, SAPO-35, SAPO-37, SAPO-44, SAPO-56, metal containing forms thereof and intergrown forms thereof.

16. The method of claim 14, wherein the activated porous metalloaluminophosphate molecular sieve comprises at least one phase of intergrown molecular sieves of the CHA framework type and of the AEI framework type.

17. The method of claim 1, further comprising aging the activated porous molecular sieve by contacting with the agent for at least 1 hour.

18. The method of claim 1, further comprising aging the activated porous molecular sieve by contacting with the agent for at least 12 hours.

19. The method of claim 1, further comprising aging the activated porous molecular sieve by contacting with the agent for at least 24 hours.

20. The method of claim 1, wherein the agent is in the vapor phase.

21. The method of claim 1, wherein the agent is in the liquid phase.

22. The method of claim 21, further comprising the step of removing at least a portion of the liquid phase agent before the molecular sieve is used in a catalytic process.

23. The method of claim 1, wherein the activated porous molecular sieve has been obtained by calcination after synthesis of the porous molecular sieve.

24. A method for converting an oxygenate feedstock into a hydrocarbon product in which an activated porous molecular sieve having a methanol uptake index of at least 0.15 is contacted with an oxygenated feedstock under oxygenate to olefins conversion conditions, wherein, prior to contacting the feedstock, the activated porous molecular sieve has been stored in contact with an agent comprising having at least 55 wt. % aldehyde and at least 0.1 wt. % water, wherein the activated porous molecular sieve has at least 80% of its pore volume filled with the agent.

25. The method of claim 24, wherein the porous molecular sieve which is contacted with the oxygenated feedstock has a methanol uptake index of at least 0.5.

26. The method of claim 24, wherein the agent comprises at least 80 wt. % aldehyde.

27. The method of claim 24, wherein the agent comprises less than 20 wt. % water.

28. The method of claim 24, wherein the agent comprises at least 1 wt. % water.

29. The method of claim 24, wherein the agent comprises at least 5 wt. % water.

30. The method of claim 24, further comprising aging the activated porous molecular sieve by contacting with the agent for at least 1 hour.

31. The method of claim 24, further comprising aging the activated porous molecular sieve by contacting with the agent for at least 12 hours.

32. The method of claim 24, further comprising aging the activated porous molecular sieve by contacting with the agent for at least 24 hours.

33. The method of claim 24, wherein the aldehyde is an alkyl aldehyde, with the alkyl group having from 1 to 16 carbon atoms, and mixtures thereof.

34. The method of claim 24, wherein the alkyl group has from 1 to 5 carbon atoms.

35. The method of claim 24, wherein the aldehyde is acetaldehyde.

36. The method of claim 24, wherein the activated porous molecular sieve has at least 80% of its pore volume filled with the agent.

37. The method of claim 24, wherein the activated porous molecular sieve has at least 95% of its pore volume filled with the agent.

38. The method of claim 24, wherein the agent is in the vapor phase.

39. The method of claim 24, wherein the agent is in the liquid phase.

40. The method of claim 24, wherein the oxygenated feedstock that contacts the molecular sieve further comprises at least 0.1 wt. % water.

41. The method of claim 24, wherein the hydrocarbon product comprises at least 0.1 wt. % methylamines.

42. The method of claim 24, wherein the hydrocarbon product includes at least 50 wt. % of one or more olefins.

43. The method of claim 42, wherein the one or more olefins include at least 40 wt. % of ethylene and propylene.

44. The method of claim 42, further comprising the step of converting the one or more olefins to a polyolefin.

45. The method of claim 24, wherein the porous molecular sieve is an activated porous metalloaluminophosphate molecular sieve.

46. The method of claim 46, wherein the porous metalloaluminophosphate molecular sieve is selected from SAPO-11, SAPO-17, SAPO-18, SAPO-34, SAPO-35, SAPO-37, SAPO-44, SAPO-56, metal containing forms thereof and intergrown forms thereof.

47. The method of claim 46, wherein the porous metalloaluminophosphate molecular sieve comprises at least one phase of intergrown molecular sieves of the CHA framework type and of the AEI framework type.

48. The method of claim 24, wherein the feedstock is contacted with the molecular sieve in a reactor and the feedstock is fed to the reactor at a rate of at least 1 kg per hour.

49. The method of claim 24, wherein the hydrocarbon product of the activated porous molecular sieve has at least 0.1 wt. % higher primary olefin selectivity (POS) as compared to the same activated porous molecular sieve without being stored in contact with the agent.

50. The method of claim 24, wherein the hydrocarbon product of the activated porous molecular sieve has at least 1 wt. % higher primary olefin selectivity (POS) as compared to the same activated porous molecular sieve without being stored in contact with the agent.

51. The method of claim 24, wherein the hydrocarbon product of the activated porous molecular sieve has at least 3 wt. % higher primary olefin selectivity (POS) as compared to the same activated porous molecular sieve without being stored in contact with the agent.

52. A method of maintaining catalytic activity of an activated porous molecular sieve, comprising the step of:
contacting the activated porous molecular sieve with an agent for at least 24 hours, wherein said agent has at least 55 wt. % aldehyde and at least 0.1 wt. % water, wherein the activated porous molecular sieve has at least 80% of its pore volume filled with the agent.

53. The method of claim 52, wherein the aldehyde is acetaldehyde.

54. A method of maintaining catalytic activity of an activated porous molecular sieve, comprising the step of:
contacting the activated porous molecular sieve with an agent having at least 80 wt. % protective material and at least 0.1 wt. % water, wherein the protective material comprises at least 20 wt. % aldehyde, 0 to 40 wt. % alcohol, and a component having at least one of C₁ to C₁₂ hydrocarbons, and any combination thereof, wherein the activated porous molecular sieve has at least 80% of its pore volume filled with the agent.

55. The method of claim 52, wherein the activated porous metalloaluminophosphate molecular sieve comprises at least one phase of intergrown molecular sieves of the CHA framework type and of the AEI framework type.

56. The method of claim 54, wherein the aldehyde is acetaldehyde.

57. The method of claim 52, wherein the activated porous metalloaluminophosphate molecular sieve comprises at least one phase of intergrown molecular sieves of the CHA framework type and of the AEI framework type.
